(19) United States
(12) Patent Application Publication
(10) Pub. No.: US 2021/0260023 A1
(43) Pub. Date: Aug. 26, 2021

(54) PURIFICATION PROCESS FOR PREPARATION OF ERIBULIN AND INTERMEDIATES THEREOF

(71) Applicant: DR. REDDY'S LABORATORIES LIMITED, Hyderabad (IN)

(72) Inventors: Thomas MAHONEY, Ely (GB); Pieter David DE KONING, Saffron Walden (GB); Graham ANDREW MEEK, Cambridge (GB); Srinivas ACHANTA, Hyderabad (IN); Rajeev Rehani BUDHDEV, Hyderabad (IN); Philip Mark JACKSON, Milton (GB); Srinivas ORUGANTI, Hyderabad (IN); Lokeswara Rao MADIVADA, Mopidevi (Mandal) (IN)

(21) Appl. No.: 17/261,125

(22) PCT Filed: Jul. 19, 2019
(86) PCT No.: PCT/IB2019/056194

§ 371 Date: Jan. 18, 2021

(30) Foreign Application Priority Data
Jul. 20, 2018 (IN) 201841027207
Sep. 26, 2018 (IN) 201841036237

(51) Int. Cl. A61K 31/357

(52) U.S. Cl. CPC A61K 31/357

(57) ABSTRACT

The present application relate to a purification process for preparation of halichondrin B analogues such as eribulin or pharmaceutically acceptable salts thereof having less than 0.15% or substantially free or free from one or more impurities. The present application also provide acid addition salts of eribulin and process for preparation thereof.

### INTRODUCTION

Aspects of the present application relate to a process for preparation of halichondrin B analogues such as eribulin or pharmaceutically acceptable salts thereof having less than 0.15% or substantially free or free from one or more impurities.

The drug compound having the adopted name eribulin, is a synthetic analogue of halichondrin B, and is represented by structure of formula I.

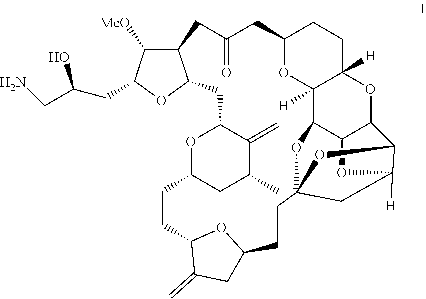

Eribulin is a microtubule inhibitor indicated for the treatment of patients with metastatic breast cancer who have previously received at least two chemotherapeutic regimens for the treatment of metastatic disease. U.S. Pat. No. 6,214,865 discloses eribulin and its pharmaceutically acceptable salts.

Processes for the preparation of Eribulin are described in U.S. Pat. No. 6,214,865, PCT publication Nos. WO 2005/118565A1, WO 2009/124237A1, WO 2015/000070A1 and WO 20151085193A1.

Some of the impurities are known to be unusually potent or to produce toxic or unexpected pharmacological effects. The US Food and Drug Administration (FDA) as well as European Medicines Agency guidance suggest that the API is free from impurities to the maximum possible extent.

The present application provides a process for the preparation of halichondrin B analogues such as eribulin or pharmaceutically acceptable salts thereof free from one or more impurities.

### SUMMARY

In the first embodiment, the present application provides eribulin or a salt thereof having less than 0.15% of one or more impurities selected from compound of formula IV or compound of formula V or compound of formula VI or isomers thereof:

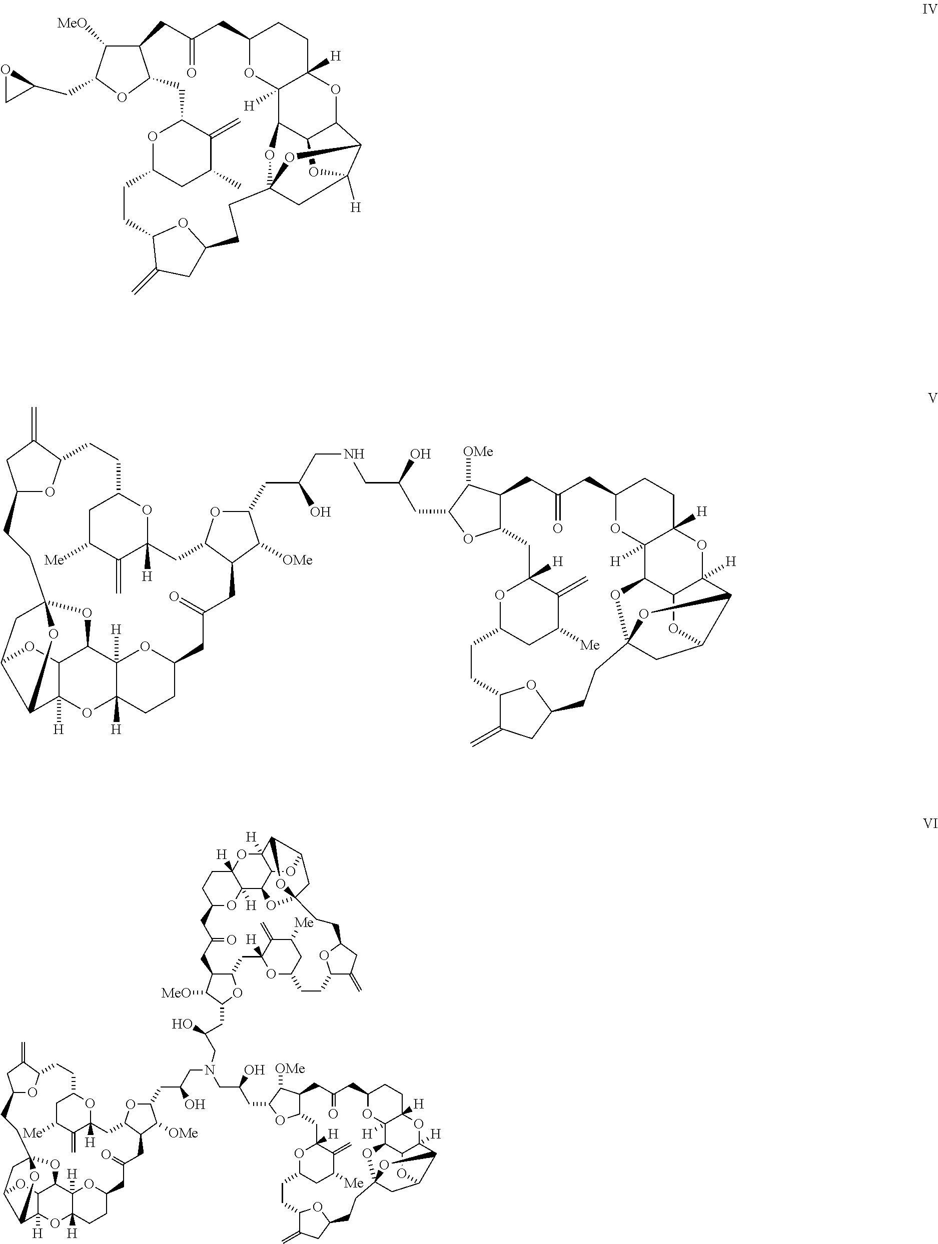

In the second embodiment, the present application provides eribulin or a salt thereof substantially free from one or more impurities selected from compound of formula IV or compound of formula V or compound of formula VI or isomers thereof.

In the third embodiment, the present application provides eribulin or a salt thereof free from one or more impurities selected from compound of formula IV or compound of formula V or compound of formula VI or isomers thereof:

In the fourth embodiment, the present application provides a purification process for preparation of eribulin or pharmaceutically acceptable salt thereof having less than 0.15% of one or more impurities selected from compound of formula IV or compound of formula V or compound of formula VI or isomers thereof, said process comprising:
(a) purifying the crude eribulin using one or more methods selected from isolation, slurrying in a suitable solvent, acid-base treatment, liquid-liquid extraction, chromatography and treating with adsorbents,
(b) converting the eribulin to eribulin pharmaceutically acceptable salt, and
(c) optionally purifying the eribulin pharmaceutically acceptable salt.

In the fifth embodiment, the present application provides a purification process for preparation of eribulin or pharmaceutically acceptable salt thereof substantially free from one or more impurities selected from compound of formula IV or compound of formula V or compound of formula VI or isomers thereof, said process comprising:
(a) purifying the crude eribulin using one or more methods selected from isolation, slurrying in a suitable solvent, acid-base treatment, liquid-liquid extraction, chromatography and treating with adsorbents,
(b) converting the eribulin to eribulin pharmaceutically acceptable salt, and
(c) optionally purifying the eribulin pharmaceutically acceptable salt.

In the sixth embodiment, the present application provides a purification process for preparation of eribulin or pharmaceutically acceptable salt thereof free from one or more impurities selected from compound of formula IV or compound of formula V or compound of formula VI or isomers thereof: said process comprising:
(a) purifying the crude eribulin using one or more methods selected from isolation, slurrying in a suitable solvent, acid-base treatment, liquid-liquid extraction, chromatography and treating with adsorbents,
(b) converting the eribulin to eribulin pharmaceutically acceptable salt, and
(c) optionally purifying the eribulin pharmaceutically acceptable salt.

In the seventh embodiment, the present application provides a purification process for preparation of eribulin or pharmaceutically acceptable salt thereof having less than 0.15% of one or more impurities selected from compound of formula IV or compound of formula V or compound of formula VI or isomers thereof, said process comprising:
(a) treating crude eribulin with a suitable reagent to provide compound of formula (VII)

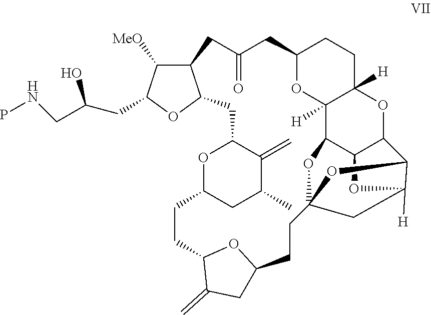

wherein P is an amine protecting group,
(b) optionally purifying compound of formula (VII) obtained in step (a),
(c) deprotecting the compound of formula (VII) to provide eribulin, and
(d) optionally converting the eribulin to pharmaceutically acceptable salt of eribulin.

In the eighth embodiment, the present application provides a purification process for preparation of eribulin or pharmaceutically acceptable salt thereof substantially free from one or more impurities selected from compound of formula IV or compound of formula V or compound of formula VI or isomers thereof, said process comprising:
(a) treating crude eribulin with a suitable reagent to provide compound of formula (VII)

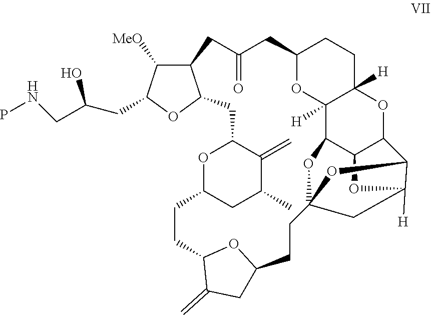

wherein P is an amine protecting group,
(b) optionally purifying compound of formula (VII) obtained in step (a),
(c) deprotecting the compound of formula (VII) to provide eribulin, and
(d) optionally converting the eribulin to pharmaceutically acceptable salt of eribulin.

In the ninth embodiment, the present application provides a purification process for preparation of eribulin or pharmaceutically acceptable salt thereof free from one or more impurities selected from compound of formula IV or compound of formula V or compound of formula VI or isomers thereof, said process comprising:
(a) treating crude eribulin with a suitable reagent to provide compound of formula (VII)

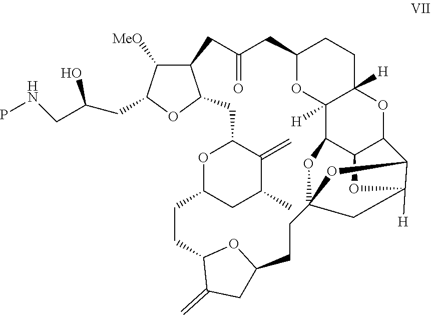

wherein P is an amine protecting group,
(b) optionally purifying compound of formula (VII) obtained in step (a),
(c) deprotecting the compound of formula (VII) to provide eribulin, and
(d) optionally converting the eribulin to pharmaceutically acceptable salt of eribulin.

In the tenth embodiment, the present application provides a process for preparation of eribulin or pharmaceutically acceptable salt thereof having less than 0.15% of one or more impurities selected from compound of formula IV or compound of formula V or compound of formula VI or isomers thereof, said process comprising:
(a) treating compound of formula (II) with sulfonylating agent to provide compound of formula (III):

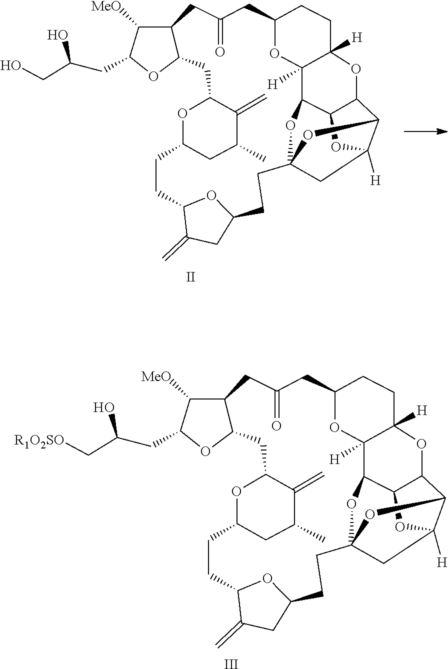

wherein R₁ is selected from straight or branched C₁-C₁₀ alkyl or optionally substituted C₅-C₁₂ aryl;
(b) converting compound of formula (III) to compound of formula (IV) and eribulin in sequence:

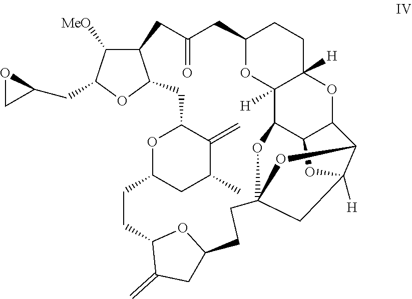

(c) purifying the crude eribulin obtained from step (b),
(d) optionally converting the eribulin to eribulin pharmaceutically acceptable salt, and
(e) optionally purifying the eribulin pharmaceutically acceptable salt

In the eleventh embodiment, the present application provides a process for preparation of eribulin or pharmaceutically acceptable salt thereof substantially free from one or more impurities selected from compound of formula IV or compound of formula V or compound of formula VI or isomers thereof, said process comprising:
(a) treating compound of formula (II) with sulfonylating agent to provide compound of formula (III):

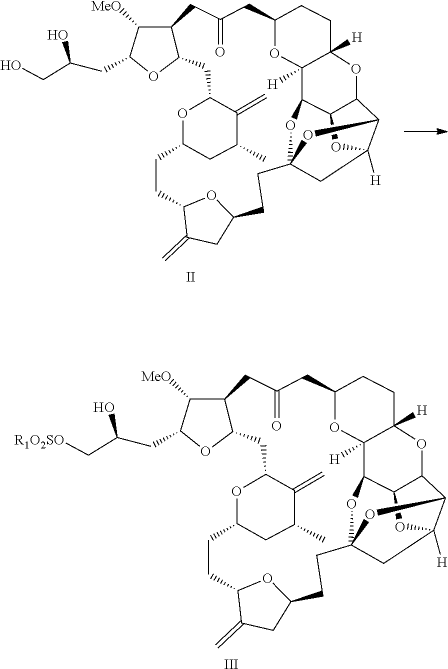

wherein R₁ is selected from straight or branched C₁-C₁₀ alkyl or optionally substituted C₅-C₁₂ aryl;
(b) converting compound of formula (III) to compound of formula (IV) and eribulin in sequence:

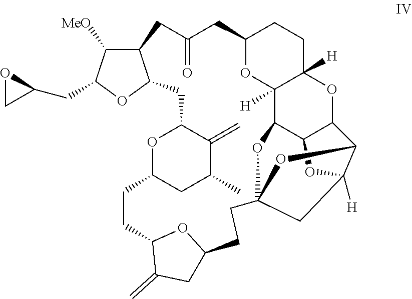

(c) purifying the crude eribulin obtained from step (b),
(d) optionally converting the eribulin to eribulin pharmaceutically acceptable salt, and
(e) optionally purifying the eribulin pharmaceutically acceptable salt

In the twelfth embodiment, the present application provides a process for preparation of eribulin or pharmaceutically acceptable salt thereof free from one or more impurities selected from compound of formula IV or compound of formula V or compound of formula VI or isomers thereof, said process comprising:
(a) treating compound of formula (II) with sulfonylating agent to provide compound of formula (III):

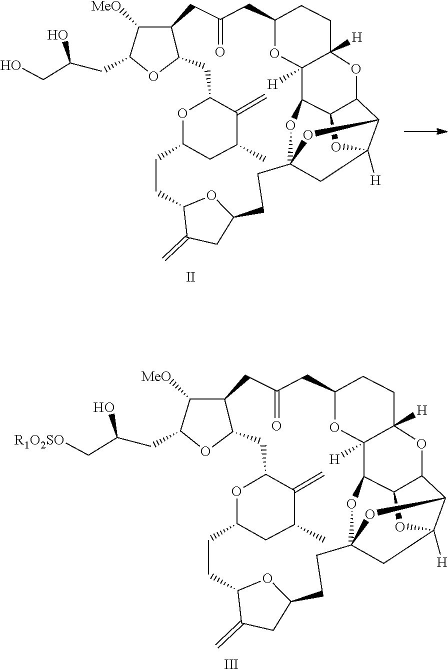

wherein R₁ is selected from straight or branched C₁-C₁₀ alkyl or optionally substituted C₅-C₁₂ aryl;
(b) converting compound of formula (III) to compound of formula (IV) and eribulin in sequence:

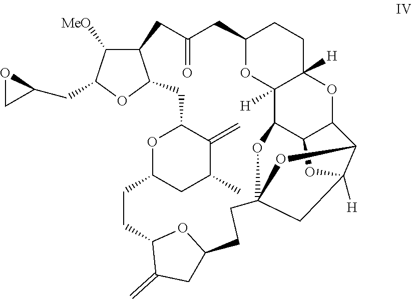

(c) purifying the crude eribulin obtained from step (b),
(d) optionally converting the eribulin to eribulin pharmaceutically acceptable salt, and
(e) optionally purifying the eribulin pharmaceutically acceptable salt

In the thirteenth embodiment, the present application provides a process for preparation of eribulin or pharmaceutically acceptable salt thereof having less than 0.15% of one or more impurities selected from compound of formula IV or compound of formula V or compound of formula VI or isomers thereof, said process comprising:
(a) treating compound of formula (II) with sulfonylating agent to provide compound of formula (III):

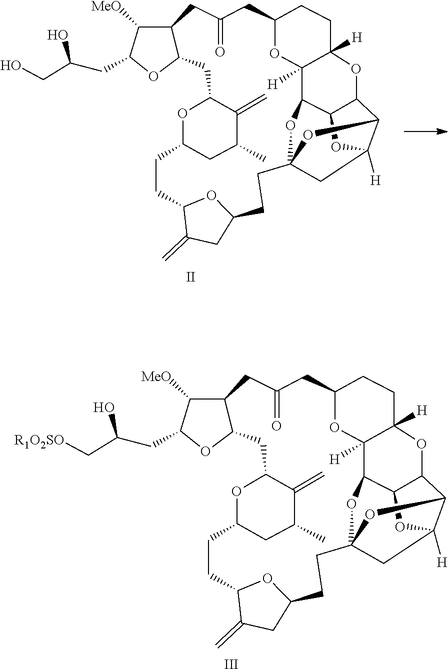

wherein R₁ is selected from straight or branched C₁-C₁₀ alkyl or optionally substituted C₅-C₁₂ aryl;
(b) converting compound of formula (III) to compound of formula (VIII) and eribulin in sequence:

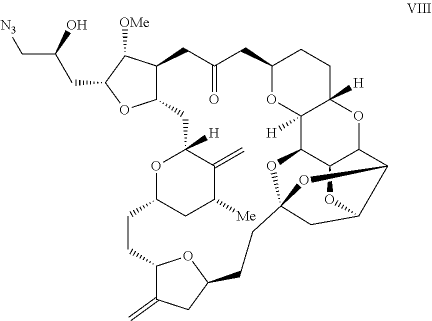 [SMILES: C=C1C[C@@H]2CC[C@@]34C[C@H]5O[C@@H]6C(O[C@H]7CC[C@H](CC(=O)C[C@@H]8[C@@H](OC)[C@@H](C[C@H](O)CN=[N+]=[N-])O[C@H]8C[C@H]8O[C@@H](CC[C@@H]1O2)C[C@@H](C)C8=C)O[C@@H]7[C@@H]6O3)[C@H]5O4]

(c) purifying the crude eribulin obtained from step (b),
(d) optionally converting the eribulin to eribulin pharmaceutically acceptable salt, and
(e) optionally purifying the eribulin pharmaceutically acceptable salt.

In the fourteenth embodiment, the present application provides a process for preparation of eribulin or pharmaceutically acceptable salt thereof substantially free from one or more impurities selected from compound of formula IV or compound of formula V or compound of formula VI or isomers thereof, said process comprising:
(a) treating compound of formula (II) with sulfonylating agent to provide compound of formula (III):

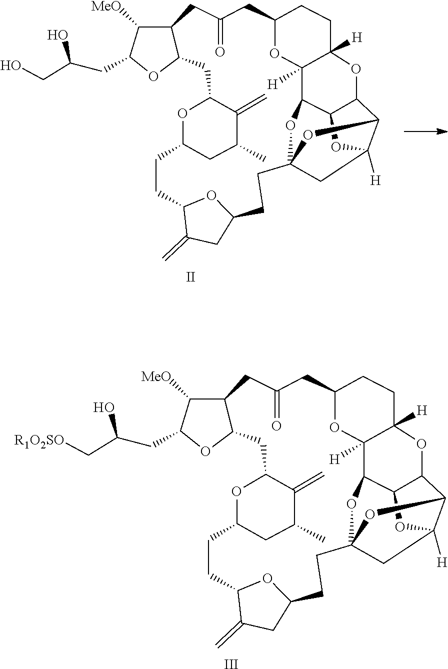

wherein R₁ is selected from straight or branched C₁-C₁₀ alkyl or optionally substituted C₅-C₁₂ aryl;
(b) converting compound of formula (III) to compound of formula (VIII) and eribulin in sequence:

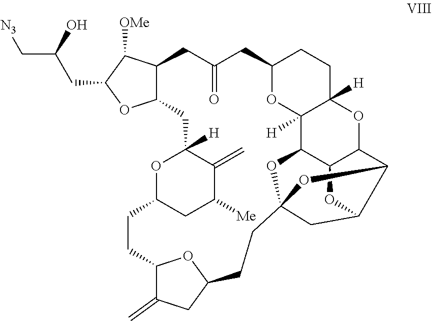 [SMILES: C=C1C[C@@H]2CC[C@@]34C[C@H]5O[C@@H]6C(O[C@H]7CC[C@H](CC(=O)C[C@@H]8[C@@H](OC)[C@@H](C[C@H](O)CN=[N+]=[N-])O[C@H]8C[C@H]8O[C@@H](CC[C@@H]1O2)C[C@@H](C)C8=C)O[C@@H]7[C@@H]6O3)[C@H]5O4]

(c) purifying the crude eribulin obtained from step (b),
(d) optionally converting the eribulin to eribulin pharmaceutically acceptable salt, and
(e) optionally purifying the eribulin pharmaceutically acceptable salt.

In the fifteenth embodiment, the present application provides a process for preparation of eribulin or pharmaceutically acceptable salt thereof free from one or more impurities selected from compound of formula IV or compound of formula V or compound of formula VI or isomers thereof, said process comprising:
(a) treating compound of formula (II) with sulfonylating agent to provide compound of formula (III):

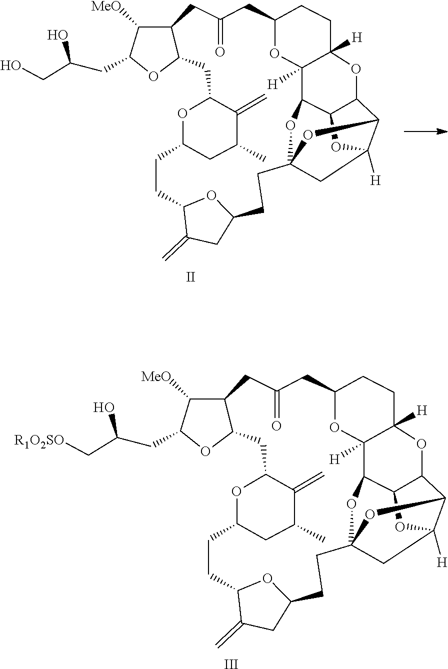

wherein R₁ is selected from straight or branched C₁-C₁₀ alkyl or optionally substituted C₅-C₁₂ aryl;
(b) converting compound of formula (III) to compound of formula (VIII) and eribulin in sequence:

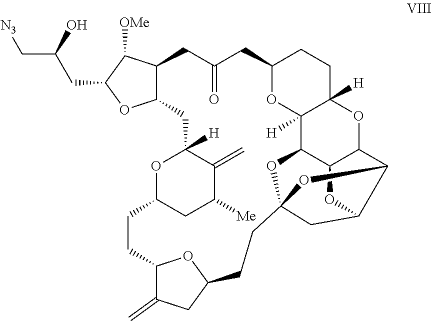 [SMILES: C=C1C[C@@H]2CC[C@@]34C[C@H]5O[C@@H]6C(O[C@H]7CC[C@H](CC(=O)C[C@@H]8[C@@H](OC)[C@@H](C[C@H](O)CN=[N+]=[N-])O[C@H]8C[C@H]8O[C@@H](CC[C@@H]1O2)C[C@@H](C)C8=C)O[C@@H]7[C@@H]6O3)[C@H]5O4]

(c) purifying the crude eribulin obtained from step (b),
(d) optionally converting the eribulin to eribulin pharmaceutically acceptable salt, and
(e) optionally purifying the eribulin pharmaceutically acceptable salt.

In the sixteenth embodiment, the present application provides acid addition salt of eribulin selected from eribulin maleate, eribulin fumarate, eribulin oxalate, eribulin citrate, eribulin acetate, eribulin benzoate, eribulin butyrate, eribulin benzenesulfonate, eribulin p-toluenesulfonate and eribulin triflate.

In the seventeenth embodiment, the present application provides compound of formula (VII) or salts thereof.

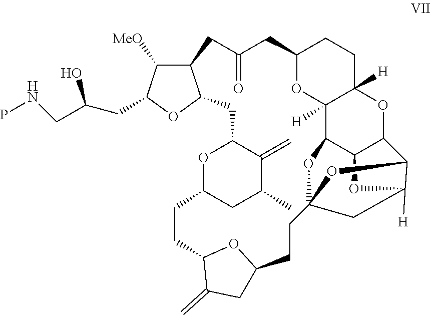

wherein P is an amine protecting group

In the eighteenth embodiment, the present application provides compound of formula VI or its pharmaceutically acceptable salts or isomers thereof.

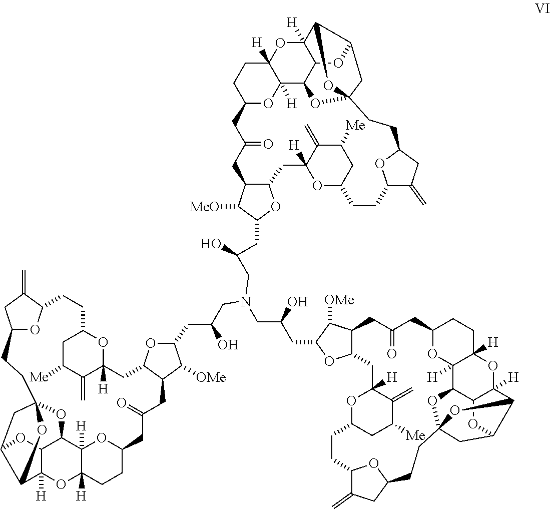

### DETAILED DESCRIPTION

In another aspect, the present application provides purification of crude eribulin of formula I or a pharmaceutically acceptable salt thereof. Purification of crude eribulin or a pharmaceutically acceptable salt thereof may be carried out by one or more methods selected from isolation, slurrying in a suitable solvent, acid-base treatment, liquid-liquid extraction, chromatography and treating with adsorbents.

Suitable isolation methods that may be used for purification of crude eribulin or a pharmaceutically acceptable salt thereof include decantation or filtration or precipitation from a solvent or precipitation by adding an anti-solvent to a solution or by evaporation of solution and the like or any other suitable isolation techniques known in the art. Optionally the said precipitation may result in a crystalline compound including solvates and hydrates thereof. Suitable solvents that may be used for said isolation include water, alcohols, ketones, hydrocarbons, halogenated hydrocarbons, esters, ethers, polar aprotic solvents, nitriles or any mixtures thereof.

Suitable solvents that may be used for purification of crude eribulin or a pharmaceutically acceptable salt thereof by slurrying in a suitable solvent include water, alcohols, ketones, hydrocarbons, halogenated hydrocarbons, esters, ethers, polar aprotic solvents, nitriles or any mixtures thereof.

Purification of crude eribulin or a pharmaceutically acceptable salt thereof may be carried out using acid-base treatment. Acid-base treatment may be carried out by treating eribulin or a pharmaceutically acceptable salt thereof with a suitable acid or a suitable base to form respective acid or base addition salt of the eribulin. The resultant acid or base addition salts of the eribulin may be optionally purified by recrystallization or washing with a suitable solvent or slurrying in a suitable solvent. The resulting acid or base addition salts of the eribulin is optionally treated with suitable desaltification agents to get the eribulin or pharmaceutically acceptable salt thereof substantially free from one or more impurities. Suitable solvents that may be used for acid-base treatment include water, alcohols, ketones, hydrocarbons, halogenated hydrocarbons, esters, ethers, polar aprotic solvents, nitriles or any mixtures thereof.

Purification of crude eribulin or a pharmaceutically acceptable salt thereof may be carried out by liquid-liquid extraction. In the said process, the compound is dissolved in a suitable first solvent to obtain a solution and the resulting solution is washed with a second solvent that is immiscible with the solution and the pure compound is isolated from the solution obtained after said washing.

Suitable chromatographic techniques that may be used for purification of crude eribulin or a pharmaceutically acceptable salt thereof selected from column chromatography, flash chromatography, ion exchange chromatography, supercritical fluid chromatography, high performance liquid chromatography (both reverse phase and normal phase), expanded bed adsorption chromatography and simulated moving bed chromatography or a combination thereof.

Suitable solvents that may be used in the chromatographic techniques include water, alcohols, ketones, hydrocarbons, halogenated hydrocarbons, esters, ethers, polar aprotic solvents, nitriles or any mixtures thereof.

Suitable mobile phases including buffers such as trifluoroacetate, sulfonate, phosphate, chloroacetate, formate, acetate, ammonium formate, ammonium bicarbonate, borate, Potassium hydrogen phosphate and the like or supercritical gases such as carbon dioxide (CO₂), xenon (Xe), nitrous oxide (N₂O), sulfur hexafluoride (SF₆), ammonia (NH₃), water (H₂O), ethane (C₂H₆), propane (C₃H₈), n-butane (C₄H₁₀) and the like in combination with suitable solvents as outlined above may be used in chromatography techniques for separation of impurities from the crude compounds which in turn give rise to pure compounds.

The chromatographic methods (for example HPLC, UPLC, and the like) that may be followed to measure the purity of eribulin or a pharmaceutically acceptable salt thereof or purification of eribulin or a pharmaceutically acceptable salt thereof involve the use of columns selected from Torus, Restek Biphenyl, YMC Pro C18, Princeton Diol, Acquity CSH Phenyl Hexyl, ZORBAX Rx-SIL, ACE 3 C18, Waters X-Bridge C18 or any other suitable chromatography columns. For example, the following methods may be used to measure the purity of eribulin or pharmaceutically acceptable salt:

HPLC method-1: Column: ACE C18-300; wave length: 200 nm; concentration: 0.5 mg/mL; diluent: water: ethanol (95:5), Flow rate: 0.5 mL/minute; Mobile phase: water, acetonitrile and IPA)

HPLC method-2: Column: waters X-Bridge C18; wave length: 200 nm; concentration: 0.5 mg/mL; diluent: water: ethanol (95:5), Flow rate: 1 mL/minute; Mobile phase: water, acetonitrile and Methanol

UPLC method: Column: Acquity Peptide CSH 150×2.1 mm×1.7 μm (Waters); Flow rate: 0.3 Ml/minute; Injection: 1.0 μL; Mobile phase: 0.1% v/v H₃PO₄ (aq) and Acetonitrile; Detector: Diode Array-single channel 200 nm

Suitable mobile phases and suitable gradient programs may be used depending on the specific impurity that needs to be separated.

Suitable resins that may be used as adsorbents in the chromatographic techniques include cation exchange resins, anion exchange resins, chelated resins, synthetic adsorbents, non-ionic resins or combinations thereof. The resins may be lipophilic, hydrophilic and/or hydrophobic in nature.

Purification of crude eribulin or a pharmaceutically acceptable salt thereof may be carried out by treating with adsorbents in a batch mode. Suitable adsorbents that may be used for purification of compounds provided in the first and second embodiments include silica gel, activated alumina, molecular sieves, magnesium silicate, synthetic resin, and the like; or any other suitable adsorbents known in the art.

The purification process may be carried out one or more times using one or more purification methods described in the present application to completely remove the impurities or to get the desired purity of eribulin or pharmaceutically acceptable salt thereof.

In an aspect suitable amine protecting groups that may be used to produce compound of formula (VII) include fluorenylmethoxycarbonyl (Fmoc), tert-butoxycarbonyl (t-BOC), benzyloxycarbonyl (Z), allyloxycarbonyl (Alloc), 2-trimethylsilylethoxycarbonyl (Teoc), 2,2,2-trichloroethoxycarbonyl (Troc), 2-trimethylsilylethylsulfonyl (SES), benzoyl, trichloroacetyl, dichloroacetyl, chloroacetyl, trifluoroacetyl, p-toluenesulfonyl, p-nitrophenylsulfonyl, aryl and alkylphosphoryl, phenyl and benzyl sulfonyl, o-nitrophenylsulfenyl, o-nitrophenoxyacetyl or any other amine protecting agents known in the art. Examples of such known amine-protecting agents are found in T. W. Green, P. G. M. Wuts, “Protective Groups in Organic Synthesis, Third Edition,” Wiley-Interscience, New York, pages 494-653, 1999.

In another aspect suitable deprotection techniques that may be used to deprotect compound of formula (VII) to produce eribulin include, catalytic hydrogenation using hydrogen gas in the presence of a metal, including Raney nickel, palladium on carbon, and the like; or hydrolysis using an acid or base; or with a fluoride source (e.g. tetra-n-butylammonium fluoride); or with any other suitable deprotection techniques known in the art. Optionally, catalytic hydrogenation may be carried out in the presence of one or more suitable reagents. Suitable reagents that may be used include, but are not limited to, acids, bases, resins, and any mixtures thereof, either alone or as their solutions in water, organic solvents or their mixtures. Suitable solvents that may be used for deprotecting compound of formula (VII) include water, alcohols, ethers, aliphatic and alicyclic hydrocarbons, halogenated hydrocarbons, aromatic hydrocarbons, nitriles, polar aprotic solvents, nitromethane or mixtures thereof.

In another aspect, the present application provides purification of compound of formula (VII). Purification of compound of formula (VII) may be carried out by one or more methods selected from isolation, slurrying in a suitable solvent, acid-base treatment, liquid-liquid extraction, chromatography and treating with adsorbents.

In an aspect, suitable sulfonylating agent that may be used for converting compound of formula (II) to compound of formula (III) include Methanesulfonyl chloride, p-Toluenesulfonyl chloride, p-Toluenesulfonic anhydride, Methanesulfonic anhydride, Trifluoro methanesulfonic anhydride, 2,4,6-Triisopropylbenzenesulfonyl chloride and the like or any other suitable sulfonylating reagents known in the art.

Compound of formula (III) is converted to compound of formula (IV) and eribulin in sequence using ammonia source. Suitable ammonia source that may be used include ammonium hydroxide or ammonia solution in a solvent like ammonia solution in THF or ammonia solution in dioxane and the like or any other ammonia sources known in the art.

### Definitions

The following definitions are used in connection with the present application unless the context indicates otherwise. In general, the number of carbon atoms present in a given group or compound is designated “Cₓ-C_{y}”, where x and y are the lower and upper limits, respectively. For example, a group designated as “C₁-C₆” contains from 1 to 6 carbon atoms. The carbon number as used in the definitions herein refers to carbon backbone and carbon branching, but does not include carbon atoms of the substituents, such as alkoxy substitutions or the like.

An “alcohol” is an organic compound containing a carbon bound to a hydroxyl group. “C₁-C₆ alcohols” include methanol, ethanol, 2-nitroethanol, 2-fluoroethanol, 2,2,2-trifluoroethanol, hexafluoroisopropyl alcohol, ethylene glycol, 1-propanol, 2-propanol (isopropyl alcohol), 2-methoxyethanol, 1-butanol, 2-butanol, i-butyl alcohol, t-butyl alcohol, 2-ethoxyethanol, diethylene glycol, 1-, 2-, or 3-pentanol, neo-pentyl alcohol, t-pentyl alcohol, cyclohexanol, phenol, glycerol and the like.

A “hydrocarbon solvent” is a liquid hydrocarbon compound, which may be linear, branched, or cyclic and may be saturated or have as many as two double bonds or aromatic. Examples of “C₅-C₁₅ aliphatic or aromatic hydrocarbons” include n-pentane, isopentane, neopentane, n-hexane, isohexane, 3-methylpentane, 2,3-dimethylbutane, neohexane, n-heptane, isoheptane, 3-methylhexane, neoheptane, 2,3-dimethylpentane, 2,4-dimethylpentane, 3,3-dimethylpentane, 3-ethylpentane, 2,2,3-trimethylbutane, n-octane, isooctane, 3-methylheptane, neooctane, cyclohexane, methylcyclohexane, cycloheptane, petroleum ethers, benzene toluene, ethylbenzene, m-xylene, o-xylene, p-xylene, indane, naphthalene, tetralin, trimethylbenzene, chlorobenzene, fluorobenzene, trifluorotoluene, anisole, C₆-C₁₂ aromatic hydrocarbons and the like.

An “ether” is an organic compound containing an oxygen atom —O— bonded to two other carbon atoms. “C₂-C₆ ethers” include diethyl ether, diisopropyl ether, methyl t-butyl ether, glyme, diglyme, tetrahydrofuran, 2-methyltetrahydrofuran, 1, 4-dioxane, dibutyl ether, dimethylfuran, 2-methoxyethanol, 2-ethoxyethanol, anisole and the like.

A “halogenated hydrocarbon” is an organic compound containing a carbon bound to a halogen. Halogenated hydrocarbons include dichloromethane, 1,2-dichloroethane, trichloroethylene, perchloroethylene, 1,1,1-trichloroethane, 1,1,2-trichloroethane, chloroform, carbon tetrachloride and the like.

An “ester” is an organic compound containing a carboxyl group —(C═O)—O— bonded to two other carbon atoms. “C₃-C₁₀ esters” include ethyl acetate, n-propyl acetate, n-butyl acetate, isobutyl acetate, t-butyl acetate, ethyl formate, methyl acetate, methyl propanoate, ethyl propanoate, methyl butanoate, ethyl butanoate and the like.

A “ketone” is an organic compound containing a carbonyl group —(C═O)— bonded to two other carbon atoms. “C₃-C₁₀ ketones” include acetone, ethyl methyl ketone, diethyl ketone, methyl isobutyl ketone, ketones and the like.

A “nitrile” is an organic compound containing a cyano —(C≡N) bonded to another carbon atom. “C₂-C₆ Nitriles” include acetonitrile, propionitrile, butanenitrile and the like.

A “polar aprotic solvents” include N, N-dimethylformamide, N, N-dimethylacetamide, dimethylsulfoxide, sulfolane, N-methylpyrrolidone and the like;

“Free from” as used herein refers to a compound that is having one or more individual impurities below its limit of detection or not detected as measured by HPLC method or UPLC method or any other analytical method. The value of limit of detection depends on the analytical method used to detect or quantify the one or more impurities.

“Substantially free” as used herein refers to a compound that is having one or more individual impurities less than about 0.05% or less than about 0.01% or less than about 0.001% or less than about 0.0001% or less than about 0.00001% or less than about 0.000001% as measured by HPLC method or UPLC method or any other analytical method.

Certain specific aspects and embodiments of the present application will be explained in greater detail with reference to the following examples, which are provided only for purposes of illustration and should not be construed as limiting the scope of the application in any manner. Reasonable variations of the described procedures are intended to be within the scope of the present application. While particular aspects of the present application have been illustrated and described, it would be obvious to those skilled in the art that various other changes and modifications can be made without departing from the spirit and scope of the invention. It is therefore intended to cover in the appended claims all such changes and modifications that are within the scope of this application.

### EXAMPLES

### Example-1

### Preparation of Eribulin

Pyridine (103 μl of a 0.33M solution prepared in dichloromethane under nitrogen) and collidine (350 μl) were added to a solution of compound of formula II (484 mg) in dichloromethane (9.1 mL) under nitrogen at −20 to −10° C. A solution of toluenesulfonic anhydride (238 mg dissolved in 5 mL dichloromethane under nitrogen) was added slowly at −10° C. and the resulting mixture was stirred for 90 minutes at −20 to −10° C. Water (1.9 mL) was added and the solution was warmed to room temperature. IPA (48 mL) and ammonium hydroxide (53 mL of a 28% solution) was added and the mixture was stirred for 23 hours. Further ammonium hydroxide (5.3 mL of a 28% solution) was added and the mixture was stirred for 17 hours. The resultant mixture was concentrated in vacuo to a total volume of ˜30 mL. Dichloromethane (15 ml) and a mixture of sodium bicarbonate/sodium carbonate/water (9:9:182 w/w/w, 4.5 g) were added. The phases were separated and the aqueous phase was re-extracted with dichloromethane (12 mL) and the combined organic phase was concentrated in vacuo at <30° C. Acetonitrile (6 mL) was added to the residue and concentrated in vacuo at <30° C. The residue was purified by flash column chromatography (methanol/dichloromethane; methanol/dichloromethane/28% ammonium hydroxide) to afford title compound (352 mg).

The obtained compound was dissolved in dichloromethane (20 mL) and washed with sodium bicarbonate/sodium carbonate/water (9:9:182 w/w/w, 25 mL). The aqueous phase was re-extracted with dichloromethane (10 mL). The combined organic extracts were concentrated in vacuo at <30° C. The residue was nitrogen purged and then dissolved in anhydrous dichloromethane/pentane (3:1 v/v, 8.5 mL) prepared under nitrogen. The resulting solution was filtered via a nitrogen-flushed cannula under nitrogen and then washed with in anhydrous dichloromethane/pentane (3:1 v/v, 8.5 mL). The filtrate was concentrated in vacuo at <30° C. The residue was dissolved in acetonitrile (2 mL) and concentrated in vacuo at <35° C. to provide the title compound as a glassy white solid (311 mg, 65%).

### Example-2

### Purification of Eribulin

A concentrated solution of crude eribulin in methanol was purified using supercritical fluid chromatography on a Princeton Diol column (mobile phase: Carbon dioxide and methanol; Gradient ratio of Carbon dioxide: methanol: 15% methanol to 35% methanol to 15% methanol). Fractions containing the purified eribulin were combined and concentrated to give eribulin.

The resultant purified eribulin was analyzed using a gradient HPLC method (Column: ACE C18-300; wave length: 200 nm; concentration: 0.5 mg/mL; diluent: water: ethanol (95:5), Flow rate: 05 mL/minute; Mobile phase: water, acetonitrile and IPA)

Content of compound of formula IV: Not detected;

Content of compound of formula V: Not detected;

Content of compound of formula VI: Not detected.

### Example-3

### Preparation of Eribulin Mesylate

A portion (5.33 g containing 40.1 mg methanesulfonic acid) of a stock solution prepared using methanesulfonic acid (201 mg), water (21.0 g) and 28% ammonium hydroxide (5.53 g) was added to eribulin (311 mg) in acetonitrile (3.9 mL). After 20 minutes stirring, the solution was concentrated in vacuo at <30° C. to ˜2 mL. Fresh acetonitrile (10 mL) was added and the solution was concentrated in vacuo at <30° C. to ˜1 ml. Fresh acetonitrile (10 mL) was added and the solution was concentrated in vacuo at <30° C. to dryness. This process was then repeated a further three times and for the final concentration. The residue was nitrogen purged and then dissolved in anhydrous dichloromethane/pentane (3:1 v/v, 7.3 mL) prepared under nitrogen. The resulting solution was filtered via a nitrogen-flushed cannula under nitrogen and then washed with anhydrous dichloromethane/pentane (3:1 v/v, 3 mL). The filtrate was concentrated in vacuo at <30° C. The residue was nitrogen purged and then dissolved in anhydrous dichloromethane/pentane (1:1 v/v, 7.4 mL) prepared under nitrogen. The resulting solution was filtered via a nitrogen-flushed cannula under nitrogen into anhydrous pentane (33.4 mL). The resultant suspension was stirred for 25 hours and then filtered via a nitrogen flushed PTFE cannula under vacuum/nitrogen and washed with further anhydrous pentane (15 mL). The material was dried under vacuum under nitrogen flow for 46 hours to provide the title compound.

The resultant eribulin was analyzed using the gradient HPLC method: Content of compound of formula IV: Not detected; Content of compound of formula V: Not detected; Content of compound of formula VI: Not detected.

### Example-4

### Preparation of N-Fmoc-(1S,3S,6S,9S,12S,14R,16R,18S,20R, 21R,22S,26R,29S,31R,32S,33R,35R,36S)-20-[(2S)-3-Amino-2-hydroxypropyl]-21-methoxy-14-methyl-8,15-bis(methylene)-2,19,30,34,37,39,40,41-octaoxanonacyclo [24.9.2.13,32.13,33.16,9.112,16.018,22.029,36.031,35]hentetracontan-24-one

Eribulin (60 mg) was charged to a 10 ml 2-necked round bottomed flask and inerted via 3 cycles of vacuum/nitrogen re-fill. Diethyl ether (1 mL) and dichloromethane (0.2 mL) were added. The stirred solution was placed in an ice-water bath and a solution of 9-fluorenylmethyl chloroformate (25 mg) in diethyl ether (0.7 mL) was added. The resultant mixture was warmed to room temperature and was stirred for 23 hours. The mixture was diluted with dichloromethane (4 mL) and washed with a solution of sodium carbonate:sodium bicarbonate:water (9:9:182 w:w:w, 3 mL). The aqueous phase was then re-extracted with dichloromethane (10 mL). The combined organic extracts were dried (K₂CO₃), filtered and the filtrate concentrated in vacuo. The residue was purified on silica gel using a CombiFlash automated purification system, eluting with MTBE/heptane to provide the title compound as a white solid (51 mg).

### Example-5

### Preparation of Eribulin

Ammonium hydroxide (0.5 mL of a 28-30% solution) was added to an ice-water bath cooled stirred solution of N-Fmoc-(1S,3S,6S,9S,12S,14R,16R,18S, 20R,21R,22S,26R,29S,31R,32S,33R,35R,36S)-20-[(2S)-3-Amino-2-hydroxypropyl]-21-methoxy-14-methyl-8,15-bis(methylene)-2,19,30,34,37,39,40,41-octaoxanon acyclo [24.9.2.13,32.13,33.16,9.112,16.018,22.029,36.031,35]hentetracontan-24-one (51 mg) in a mixture of iso-propanol (1 mL) and dichloromethane (0.2 mL). After stirring for 67 hours, the mixture was concentrated in vacuo in a water bath at <30° C. Dichloromethane (5 mL) and sodium carbonate: sodium bicarbonate: water (9:9:182 w:w:w, 1 mL) were added to the residue. The phases were separated and the aqueous phase was re-extracted with dichloromethane (5 mL). The combined organic extracts were concentrated in vacuo in a water bath at <30° C. The residue was purified by flash column chromatography eluting with MTBE/MeOH/dichloromethane/ammonium hydroxide to provide the title compound.

The resultant eribulin was analyzed using the gradient HPLC method: Content of compound of formula IV: Not detected; Content of compound of formula V: Not detected; Content of compound of formula VI: Not detected.

### Example-6

### Preparation of eribulin maleate salt

Crude eribulin (115 mg having purity 94.8% by UPLC) was dissolved in ethyl acetate (0.5 mL) and stirred at room temperature. A solution of maleic acid (16.5 mg) in ethyl acetate (0.5 mL) was added and the mixture was stirred for 1 hour at room temperature. The separated solid was filtered, washed with ethyl acetate (5 mL) and dried under vacuum with nitrogen flow to afford title compound.

Purity by UPLC: 98.8%; content of compound of formula IV: Not detected; content of compound of formula V: Not detected; content of compound of formula VI: Not detected.

### Example-7

### Preparation of Eribulin Oxalate Salt

Crude eribulin (104 mg having purity 94.8% by UPLC) was dissolved in ethyl acetate (0.5 mL) and stirred at room temperature. A solution of oxalic acid (12.1 mg) in ethyl acetate (0.5 mL) was added and the mixture was stirred for 1 hour at room temperature. The solvent was evaporated to give a white solid which was triturated with MTBE (2 mL). The solid was filtered, washed with MTBE (3×2 mL) and dried under vacuum with nitrogen flow to give title compound.

Purity by UPLC: 98.4%; content of compound of formula IV: Not detected; content of compound of formula V: Not detected; content of compound of formula VI: Not detected.

### Example-8

### Preparation of Eribulin Fumarate Salt

Crude eribulin (105 mg having purity 94.8% by UPLC) was dissolved in methanol (0.5 mL) and stirred at room temperature. A solution of fumaric acid (16 mg) in methanol (0.5 mL) was added and the mixture was stirred for 1 hour at room temperature. The solvent was evaporated and MTBE (1 mL) was added. The mixture was stirred for 30 minutes and the separated solid was filtered, washed with MTBE (3×2 mL) and dried under vacuum with nitrogen flow to title compound.

Purity by UPLC: 98.2%; content of compound of formula IV: Not detected; content of compound of formula V: Not detected; content of compound of formula VI: Not detected.

### Example-9

### Preparation of Eribulin Tosylate Salt

Crude eribulin (136 mg having purity of 94.8% by UPLC) was dissolved in acetonitrile (2.5 mL). A solution of toluenesulfonic acid monohydrate (34.7 mg) in 28% aqueous ammonium hydroxide (1.95 mL) and water (0.5 mL) was added. The mixture was concentrated under reduced pressure at 25-30° C. The residue was azeotroped from acetonitrile (8×3 mL) to give a white foam (188 mg). The foam was dissolved in DCM/pentane (3:1, v/v, 3 mL) and filtered, washing with DCM/pentane (3:1, v/v, 2×1 mL) to give a clear solution which was concentrated to a white foam (202 mg) and then dissolved in DCM/pentane (1:1, v/v, 2 mL). The solution was added to pentane (15 mL) with stirring at room temperature under nitrogen. An immediate precipitate was observed. The residues were washed in with DCM/pentane (1:1, v/v, 2×0.25 mL). The slurry was stirred for 24 h and then filtered through an enclosed filter under nitrogen. The solid was washed with pentane (6.5 mL) and dried under vacuum with nitrogen flow overnight to give the tosylate salt as a white solid Analyzed using UPLC: content of compound of formula IV: Not detected; content of compound of formula V: Not detected; content of compound of formula VI: Not detected.

### Example-10

### Purification of Eribulin

Crude eribulin was purified using reversed phase preparative UPLC (Column: Acquity CSH C18 column; gradient method; Mobile phases: Aqueous formic acid and acetonitrile). The relevant fractions were collected, combined and concentrated to dryness on a rotary evaporator to afford eribulin.

The resultant eribulin was analyzed using the gradient HPLC method: Content of compound of formula IV: Not detected; Content of compound of formula V: Not detected; Content of compound of formula VI: Not detected.

1. Eribulin or a pharmaceutically acceptable salt thereof having less than 0.15% of one or more impurities selected from compound of formula IV, compound of formula V and compound of formula VI or isomers thereof.

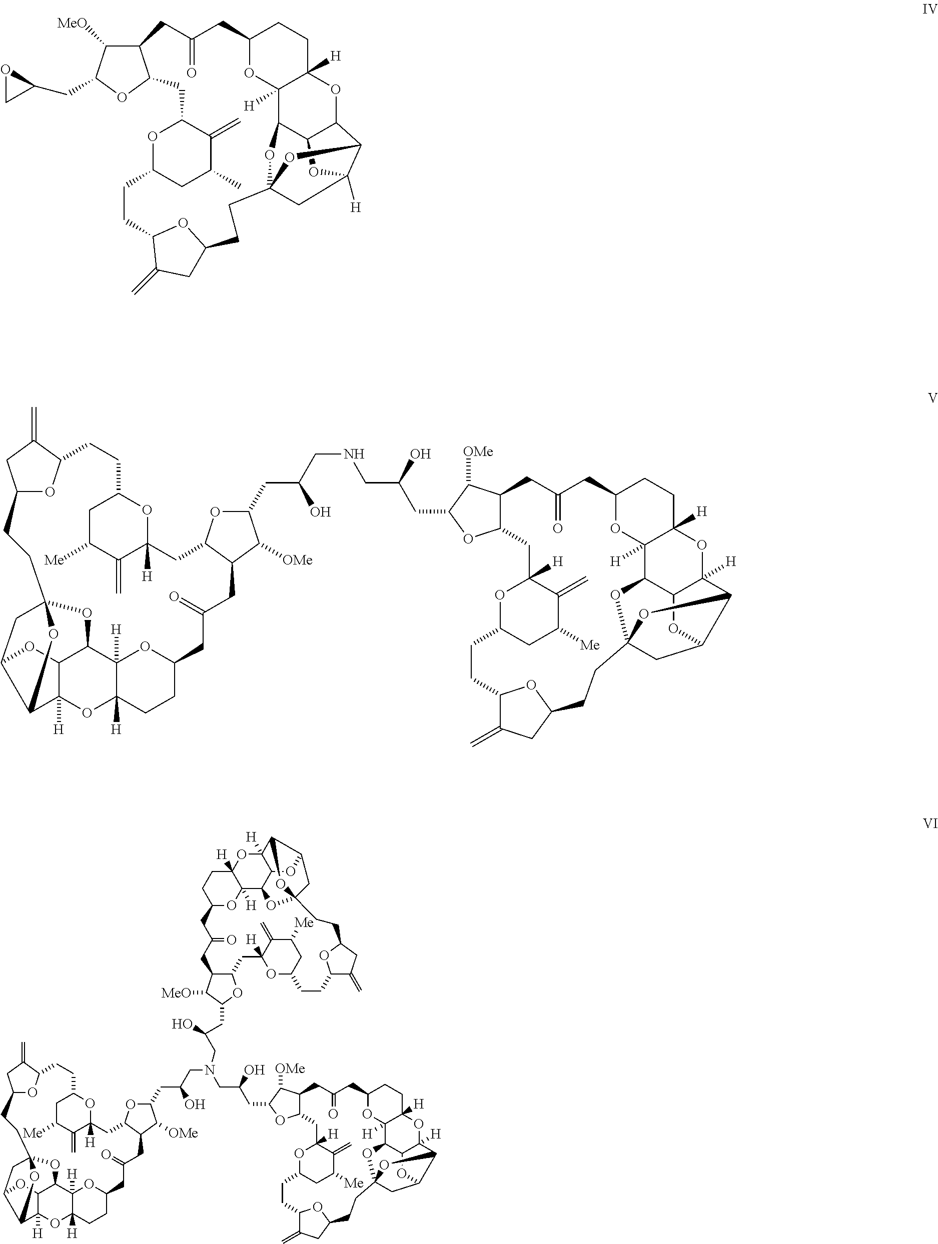

2. The compound of claim 1, wherein eribulin or its pharmaceutically acceptable salt is substantially free from one or more impurities selected from compound of formula IV, compound of formula V and compound of formula VI or isomers thereof.

3. The compound of claim 1, wherein eribulin or its pharmaceutically acceptable salt is substantially free from compound of formula IV.

4. The compound of claim 1, wherein eribulin or its pharmaceutically acceptable salt is substantially free from compound of formula V.

5. The compound of claim 1, wherein eribulin or its pharmaceutically acceptable salt is substantially free from compound of formula VI.

6. The compound of claim 1, wherein eribulin or its pharmaceutically acceptable salt is free from one or more impurities selected from compound of formula IV, compound of formula V and compound of formula VI or isomers thereof.

7. The compound of claim 1, wherein eribulin or its pharmaceutically acceptable salt is free from compound of formula IV.

8. The compound of claim 1, wherein eribulin or its pharmaceutically acceptable salt is free from compound of formula V.

9. The compound of claim 1, wherein eribulin or its pharmaceutically acceptable salt is free from compound of formula VI.

10. A process for preparation of eribulin or pharmaceutically acceptable salt thereof according to claim 1, said process comprising:
(a) purifying crude eribulin using one or more methods selected from isolation, slurrying in a suitable solvent, acid-base treatment, liquid-liquid extraction, chromatography and treating with adsorbents,
(b) converting the eribulin obtained in step (a) to eribulin pharmaceutically acceptable salt, and
(c) optionally purifying the eribulin pharmaceutically acceptable salt.

11. The process according to claim 10, wherein the crude eribulin is purified using chromatographic methods.

12. The process according to claim 11, wherein the crude eribulin is purified using flash chromatography or ion exchange chromatography or supercritical fluid chromatography or high performance liquid chromatography.

13. The process according to claim 10, wherein the crude eribulin is purified by isolating eribulin acid addition salts.

14. The process according to claim 13, wherein the acid addition salts of eribulin is prepared by treating eribulin with acids selected from maleic acid, fumaric acid, oxalic acid, citric acid, acetic acid, benzoic acid, butyric acid, benzenesulfonic acid, p-toluenesulfonic acid and trifluoroacetic acid.

15. The process according to claim 10, wherein the crude eribulin or its pharmaceutically acceptable salt is purified by isolating from one or more solvents selected from water, alcohols, ketones, hydrocarbons, halogenated hydrocarbons, esters, ethers, polar aprotic solvents and nitriles.

16. A process for preparation of eribulin or pharmaceutically acceptable salt thereof according to claim 1, said process comprising:
(a) treating crude eribulin with a suitable reagent to provide compound of formula (VII) 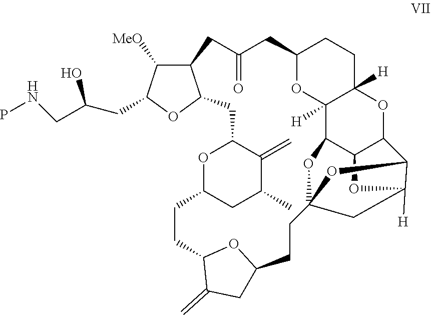
wherein P is an amine protecting group,
(b) optionally purifying compound of formula (VII) obtained in step (a),
(c) deprotecting the compound of formula (VII) to provide eribulin, and
(d) optionally converting the eribulin to pharmaceutically acceptable salt of eribulin.

17. Compound of formula (VII) or salts or isomers thereof. 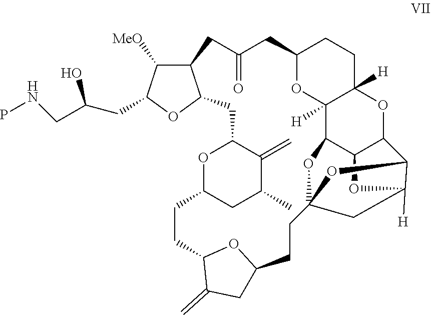
wherein P is an amine protecting group

18. Compound of formula VI or its pharmaceutically acceptable salts or isomers thereof. [chemical structure]
